(19) United States
(12) Patent Application Publication
(10) Pub. No.: US 2015/0368230 A1
(43) Pub. Date: Dec. 24, 2015

(54) RADIOLABELED QUINAZOLINE DERIVATIVES

(71) Applicants: BOEHRINGER INGELHEIM INTERNATIONAL GMBH, Ingelheim Ann Rhein (DE); STICHTING VU-VUMC, Amsterdam (NL)

(72) Inventors: Paul SLOBBE, Amstelveen (NL); Alex Johannes POOT, Vleuten (NL); Augustinus Antonius Maria Silvester VAN DONGEN, Utrecht (NL); Heiko NIESSEN, Biberach an der Riss (DE); Albert Dirk WINDHORST, Alphen aan den Rijn (NL)

(21) Appl. No.: 14/765,042

(22) PCT Filed: Jan. 28, 2014
(86) PCT No.: PCT/EP2014/051659

§ 371 Date: Jul. 31, 2015

(30) Foreign Application Priority Data
Feb. 1, 2013 (EP) 13153670.8

(51) Int. Cl. C07D 405/12; A61K 51/04

(52) U.S. Cl. CPC C07D 405/12; A61K 51/0459; C07B 2200/05

(57) ABSTRACT

Novel radiotracer for positron emission tomography (PET) or single photon emission computed tomography (SPECT) imaging are described. Specifically, the invention relates to ¹⁸F-labeled afatinib suitable as a PET or SPECT tracer for imaging epidermal growth factor receptor (EGFR, erbB1) and human epidermal growth factor receptor 2 (Her2, erbB2), to a precursor compound for use in its synthesis, to methods for the preparation ¹⁸F-labeled afatinib, as well as to the use thereof in in vivo diagnosis, tumor imaging or patient stratification on the basis of mutational status of EGFR (erbB1) and Her2 (erbB2).

### FIELD OF THE INVENTION

The present invention relates to radiolabeled compounds of formula (I)

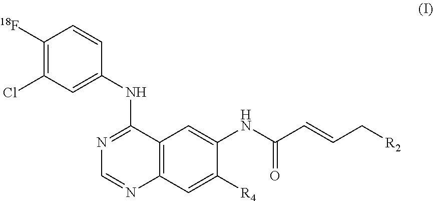

suitable as PET or SPECT tracer for imaging epidermal growth factor receptor (EGFR, erbB1) and human epidermal growth factor receptor 2 (Her2, erbB2) and their use in in vivo diagnosis, tumor imaging or cancer patient stratification on the basis of mutational status of EGFR (erbB1) and Her2 (erbB2). The present invention also describes a precursor compound and methods of preparing the radiotracer. The invention is relevant to any cancer that is influenced or driven by deregulated Human Epidermal Growth Factor Receptor (HER/Human EGFR) such as, but not limited to, non-small cell lung cancer (NSCLC), head and neck squamous cell carcinoma (HNSCC), breast cancer, esophageal cancer, gastric cancer, renal cancer, cervical cancer, ovarian cancer, pancreatic cancer, hepatocellular cancer, malignant glioma, prostate cancer and colorectal cancer (CRC).

### BACKGROUND OF THE INVENTION

Positron emission tomography (PET) and single photon emission computed tomography (SPECT) are a nuclear medicine imaging techniques that produce images of functional processes of the body. Radiotracers are used in PET or SPECT as diagnostic tools and to image tissue concentration of molecules of interest.

Compounds of formula (I) are, disclosed in WO02/50043, WO2004/074263 and WO2005/037824 as dual inhibitors of EGFR (erbB1) and Her2 (erbB2) receptor tyrosine kinases, suitable for the treatment of e.g. benign or malignant tumours, particularly tumours of epithelial and neuroepithelial origin. Pharmaceutical formulations of the compounds are disclosed in the cited documents and in WO2009/147238.

Indications to be treated and combination treatments are disclosed in WO2007/054550 and WO2007/054551.

Oncogene 2008, 4702-4711 describes irreversible inhibitors exhibiting different in vitro and in vivo potency for different types of activating mutations of the EGFR.

Lung Cancer 2012, 123-127 and Lancet Oncology 2012, 539-548 describe potent effects of irreversible inhibitors in patients harboring mutations of EGFR compared to patients that express Wild Type (WT) EGFR in lung cancer.

The aforementioned irreversible inhibitors are both active against EGFR (erbB1) mutations targeted by first generation therapies and against those not sensitive to these standard therapies.

The present invention aims to provide radioligands selective for EGFR (erbB1) and Her2 (erbB2) as PET or SPECT tracer for in vivo diagnosis or tumor imaging in patients harboring mutations of EGFR (erbB1).

### DESCRIPTION OF THE INVENTION

A first aspect of the invention is a radiolabeled compound of formula (I)

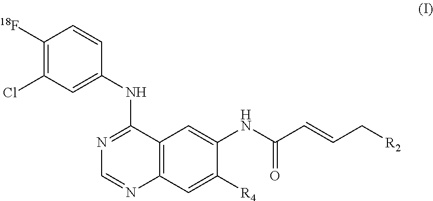

wherein
R² represents dimethylamino-, diethylamino-, morpholino-, [1,4]oxazepan-4-yl-
R⁴ represents tetrahydrofuran-3-yl-oxy-, tetrahydrofuran-2-yl-methoxy-, tetrahydrofuran-3-yl-methoxy-, tetrahydropyran-4-yl-oxy-, or tetrahydropyran-4-yl-methoxy-.

Another embodiment of the first aspect of the invention is directed to a radiolabeled compound as hereinbefore defined,

wherein
R² represents dimethylamino-.

Yet another embodiment of the first aspect of the invention is directed to a radiolabeled compound as hereinbefore defined,

wherein
R⁴ represents

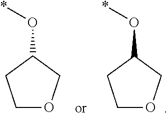

Another embodiment of the first aspect of the invention is directed specifically to the radiolabeled compounds as hereinbefore defined selected from

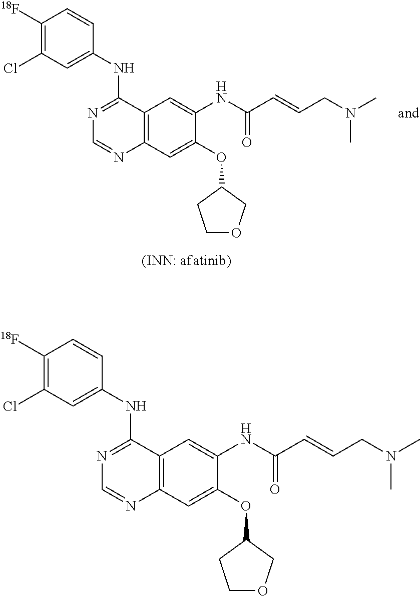

A second aspect of the invention is directed to an intermediate compound of formula (II)

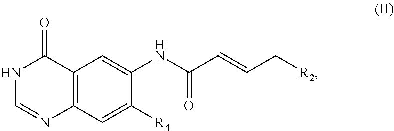

wherein
R² and R⁴ are defined as hereinbefore.

The radiolabeled compound of general formula (I) as hereinbefore defined may be prepared by the following method, for example:

reacting radiolabeled literature known

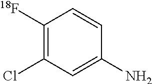

### (J Label Compd Radiopharm 2005, 48, 829-843)

with a compound of formula (II)

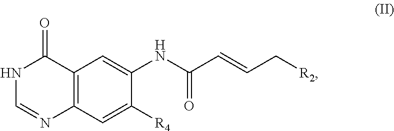

wherein
R² and R⁴ are defined as hereinbefore,
and isolating the resulting compound of formula (I).

The reaction is optionally carried out in a solvent or mixture of solvents such as N-methylpyrrolidine (NMP), acetonitrile (MeCN), tetrahydrofuran (THF), dichloromethane (DCM), N,N-dimethylformamide (DMF), dimethylsulfoxide (DMSO) or tert-butanol and optionally in the presence of an inorganic or organic base such as 2,3,4,6,7,8,9,10-octahydropyrimido[1,2-a]azepine (DBU), triethylamine, diethylisopropylamine, diisopropylamine or potassium-tert-butoxide and optionally in the presence of benzotriazol-1-yl-oxy-tris-(dimethylamino)-phosphonium hexafluorophosphate (BOP) or (benzotriazol-1-yl-oxy)tripyrrolidinophosphonium hexafluorophosphate (PyBOP) or 6-chloro-benzotriazol-1-yl-oxy-tris-pyrrolidinophosphonium hexafluorophosphate (PyClock) or bromotripyrrolidinophosphonium hexafluorophosphate (PyBrop) or 1-hydroxybenzotriazole (HOBt) or N-Ethyl-N′-(3-dimethylaminopropyl)carbodiimide (EDC) in one embodiment at temperatures between −50° C. and 250° C., in another embodiment at temperatures between 00° C. and 200° C., and in yet another embodiment between 50° C. and 150° C.

The resulting compound of formula (I) is optionally purified by chromatography, HPLC chromatography or solid phase extraction (SPE). HPLC chromatography is optionally carried out using reverse phase material as solid phase such as C18, C18-EPS or C8 and a solvent or mixture of solvents as eluent such as methanol, ethanol, acetonitrile or water and optionally in the presence of a buffer, acid or base such as ammonium dihydrogen phosphate, phosphoric acid, trifluoracetic acid or diisopropylamine. Reformulation of the purified HPLC product is required to remove solvents that are not allowed for injection into human. Therefore, solid phase extraction (SPE) is optionally carried out using solid phases such as C18, tC18, Silica and a solvent as eluent such as ethanol which is suitable for in vivo injection, when diluted to maximally 12-volume percent.

The intermediate compound of formula (II) as hereinbefore defined may be prepared by the following method, for example:

reacting a compound of formula (III)

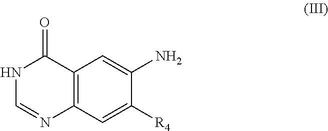

wherein
R⁴ is defined as hereinbefore,
with a compound of formula (IV)

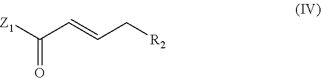

wherein
R² is defined as hereinbefore and Z₁, is a leaving group such as a halogen atom, e.g. a chlorine or a bromine atom, or a hydroxy group.

The reaction is optionally carried out in a solvent or mixture of solvents such as dichloromethane, N,N-dimethylformamide, N-methylpyrrolidine, benzene, toluene, chlorobenzene, tetrahydrofuran, benzene/tetrahydrofuran or dioxane, optionally in the presence of an inorganic or organic base and optionally in the presence of a dehydrating agent, in one embodiment at temperatures between −50° C. and 150° C., in another embodiment at temperatures between −20° C. and 80° C.

With a compound of general formula (IV) wherein Z₁, denotes a leaving group, the reaction is optionally carried out in a solvent or mixture of solvents such as dichloromethane, N,N-dimethylformamide, N-methylpyrrolidine, benzene, toluene, chlorobenzene, tetrahydrofuran, benzene/tetrahydrofuran or dioxane, conveniently in the presence of a tertiary organic base such as triethylamine, pyridine or 4-dimethylaminopyridine, in the presence of diisopropylethylamine (Hünig base), whilst these organic bases may simultaneously also act as solvent, or in the presence of an inorganic base such as sodium carbonate, potassium carbonate or sodium hydroxide solution, in one embodiment at temperatures between −50° C. and 150° C., in another embodiment at temperatures between −20° C. and 80° C.

With a compound of general formula (IV) wherein Z₁ denotes a hydroxy group, the reaction is optionally carried out in the presence of a dehydrating agent, e.g. in the presence of isobutyl chloroformate, thionyl chloride, oxalylchloride, trimethyl chlorosilane, phosphorus trichloride, phosphorus pentoxide, hexamethyldisilazane, N,N′-dicyclohexylcarbodiimide, N,N′-dicyclohexylcarbodiimide/N-hydroxysuccinimide, 1-hydroxy-benzotriazole, N,N′-carbonyldiimidazole or triphenylphosphine/carbon tetrachloride, expediently in a solvent such as dichloromethane, N-methylpyrrolidine, tetrahydrofuran, dioxane, toluene, chlorobenzene, N,N-dimethylformamide, dimethylsulphoxide, ethylene glycol diethylether or sulpholane and optionally in the presence of a reaction accelerator such as 4-dimethylaminopyridine or N,N-dimethylformamide in one embodiment at temperatures between −50° C. and 150° C., in another embodiment at temperatures between −20° C. and 80° C.

The radiolabeled compound of general formula (I) as hereinbefore defined or a physiologically acceptable salt thereof is optionally used in in vivo diagnosis, tumor imaging or patient stratification on the basis of mutational status of EGFR (erbB1). Uptake of the radiolabeled compound of general formula (I) in the mutated tumours can be determined with PET or SPECT. Examples of this principle with ¹¹C-erlotinib are published by Memon et al in British Journal of Cancer, 2011, 1850-1855 and by Bahce et al in Clinical Cancer Research, 2012, doi: 10.1158/1078-0432.CCR-12-0289 (accepted for publication). An aqueous formulation which is sterile, pyrogen free and isotonic is optionally prepared by diluting the hereinbefore mentioned ethanolic eluate with pharmaceutically acceptable buffers such as 0.9% sodium chloride, sodiumdihydrogenphosphate 7.09 mM in 0.9% sodiumchloride or citrate buffer, pharmaceutically acceptable solubilisers such as, ethanol, tween or phospholipids and/or with pharmaceutically acceptable stabilizers or antioxidants such as ascorbic acid, gentisic acid or p-aminobenzoic acid. The final formulation should contain maximally 12-volume percent of eluent. Patients are administered typically 150-500 MBq of product by intravenous injection.

The radiolabeled compound of general formula (I) can advantageously be used as diagnostic agent for imaging in vivo of EGFR (erbB1) up regulated tumors such as shown with ¹¹C-erlotinib by Memon et al in British Journal of Cancer, 2011, 1850-1855 and by Bahce et al in Clinical Cancer Research, 2013, 183-193 doi: 10.1158/1078-0432.CCR-12-0289.

The radiolabeled compound of general formula (I) can advantageously be used for the stratification of non small cell lung cancer patients since only 10-30% of the patients population is responsive to treatment with EGFR inhibitors. The radiolabeled compound of general formula (I) can be used to discriminate these patients by increased tumor accumulation of the radiotracer as determined by positron emission tomography (PET) or single photon emission computed tomography (SPECT) in vivo.

Furthermore, as example, in non small cell lung cancer (NSCLC), but not limited to NSCLC, several mutational variants of the EGF receptor are known and associated with different clinical outcome of treatment. Examples include but are not limited to, a point mutation in exon 21 of the EFG receptor (L858R) leading to increased sensitivity to small molecule tyrosine kinase inhibitors, a point mutation in exon 20 (T790M) leading to resistance to first generation tyrosine kinase inhibitors and exon 19 deletions conferring sensitivity to small molecule tyrosine kinase inhibitors. The radiolabeled compound of general formula (I) can be advantageously used to discriminate between these types of mutation as higher accumulation of the radiolabeled compound of general formula (I) as assessed by PET or SPECT is a method to define the mutational status of the tyrosine kinase in vivo.

Another option for a radiotracer for in vivo diagnosis is a radiolabeled compound of formula (V)

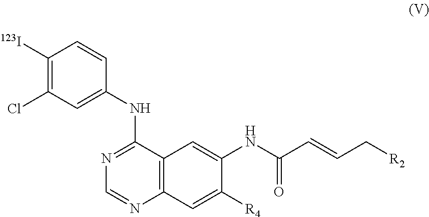

wherein
R² represents dimethylamino-, diethylamino-, morpholino-, [1,4]oxazepan-4-yl-;
R⁴ represents tetrahydrofuran-3-yl-oxy-, tetrahydrofuran-2-yl-methoxy-, tetrahydrofuran-3-yl-methoxy-, tetrahydropyran-4-yl-oxy-, or tetrahydropyran-4-yl-methoxy-.

Another option for a radiotracer for in vivo diagnosis is directed to a radiolabeled compound as hereinbefore defined,

wherein
R² represents dimethylamino-.

Yet another option for a radiotracer for in vivo diagnosis is directed to a radiolabeled compound as hereinbefore defined,

wherein
R⁴ represents

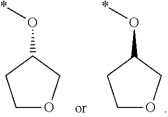

Another option for a radiotracer for in vivo diagnosis is directed specifically to the radiolabeled compounds as hereinbefore defined selected from

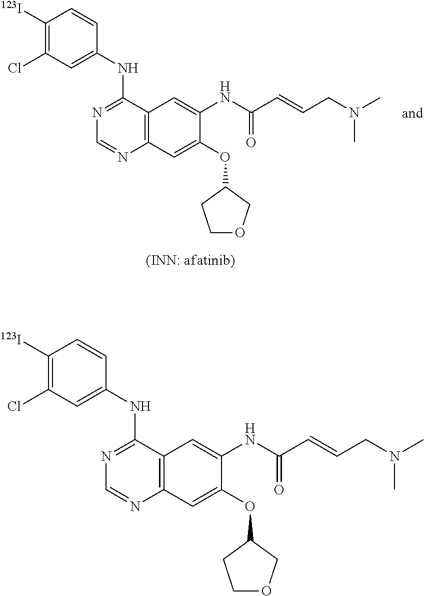

4-[¹²³I]iodo-3-choroaniline can be obtained from 3-chloroaniline by a person skilled in the art by either oxidative [¹²³I]iodination or by electrophilic [¹²³I]iodination of 4-(tetra-n-alkyl)-3-chloroaniline following literature procedures and can be reacted with compound according to formula (II) to yield a compound according to formula (V).

The invention is thus also directed to a method for the in vivo diagnosis or imaging of EGFR (erbB1) positive tumors, as well as the characterization and distribution of the mutational status of said receptor in the tumor in a subject, preferably a human, comprising administration of the above described radio labeled compound (I) according to the invention to the patient. Thereby, the invention also provides a diagnostic method for (EGFR dependant) cancer patient stratification for sensitivity to treatment with EGFR inhibitors based on molecular imaging. Administration of the compound is preferably in a radiopharmaceutical formulation comprising the compound or its salt or solvate and one or more pharmaceutically acceptable excipients in a form suitable for intra venous administration to humans. The radiopharmaceutical formulation is preferably an aqueous sterile, isotonic and pyrogen free solution additionally comprising a pharmaceutically acceptable buffer, a pharmaceutically acceptable solubiliser such as, but not limited to, ethanol, tween or phospholipids, pharmaceutically acceptable stabilizer solutions and/or antioxidants such as, but not limited to, ascorbic acid, gentisic acid or p-aminobenzoic acid. The final formulation should contain maximally 12-volume percent of eluent. Patients are administered typically 150-500 MBq of product by intravenous injection.

The invention is thus also directed to a radiopharmaceutical formulation comprising the radiolabeled compound of general formula (I), suitable for application as an in vivo diagnostic or within an imaging method, wherein the method is preferably positron emission tomography (PET) or single photon emission computed tomography (SPECT).

### SHORT DESCRIPTION OF THE FIGURES

FIG. 1 illustrates immunhistochemical staining of tumor xenografts used in PET-study.

FIG. 2 shows the biodistribution of [¹⁸F]afatinib in A549 (n=3, 6 tumors), H1975 (n=3, 6 tumors) and HCC827 (n=3, 6 tumors) tumor bearing mice. Columns show the percentage of injected dose per gram (% ID/g) per organ. Errors are standard error of the mean (SEM).

FIGS. 3 to 7 show the results of dynamic PET imaging performed on three cancer xenograft (A549, H1975 and HCC827) lines in nude mice, including a comparative imaging study with [¹¹C]erlotinib. The results are grouped per cell line. The left two panels in FIGS. 3 to 5 are the [¹⁸F]afatinib time activity curves (TAC) (top: no block, bottom: blocked with tariquidar) and the right are the [¹¹C]erlotinib scans (top: no block, bottom: blocked with tariquidar).

FIG. 3 shows the results obtained with A549—wild type.

FIG. 4 shows the results obtained with H1975—L858R/T790M—acquired resistance.

FIG. 5 shows the results obtained with HCC827—exon 19 del.

FIG. 6 relates to brain uptake of [¹⁸F]afatinib and shows that when P-gp was active [¹⁸F]afatinib is washed out from the brain and under blocking conditions remains in the brain.

FIG. 7 shows the results of imaging in the presence of varying amounts of cold afatinib added to the [¹⁸F]afatinib injection in an attempt to mimic a therapeutic dose. Already when adding a cold dose of 100 ng tumor uptake was reduced to background levels. This shows that imaging of the tumor should be done at high specific activity.

### METABOLITE ANALYSIS

Six Balb/C mice were injected with 20-30 MBq of [¹⁸F]afatinib, in the ocular plexus under isoflurane anesthesia (2% in 1 L·min⁻¹). The mice were sacrificed at 15 (n=4) and 45 (n=4) min post-injection. At these time points, about 1.5 mL of blood was collected via a heart puncture. Blood was collected in a heparin tube and centrifuged for 5 minutes at 4000 r.p.m. (Hettich universal 16, Depex B.V., the Netherlands). Plasma was separated from blood cells and 1 mL of plasma was diluted with 2 mL of 0.1 M hydrochloric acid and loaded onto a tC2 Sep-Pak cartridge, which was pre-activated by elution with 3 mL of MeOH and 6 mL of water, respectively. The cartridge was washed with 5 mL of H₂O to collect polar radioactive metabolites. Thereafter, the tC2 Sep-Pak cartridge was eluted with 2 mL of MeOH and 1 mL of H₂O to collect the mixture of apolar metabolites. The mixture of apolar metabolites was analyzed using HPLC to determine the percentage of intact [¹⁸F]afatinib. HPLC was performed on a Dionex Ultimate 3000 system, equipped with a 1 mL loop. As a stationary phase a Phenomenex Gemini C18, 250×10 mm, 5 μm was used. The mobile phase was a gradient of A=acetonitrile and B=0.1% DiPA in H₂O. The HPLC gradient ran for 12.5 minutes increasing the concentration of eluent B from 0% to 10% at a flow rate of 4 ml·min⁻¹. Resulting in the metabolic profile listed in table 1, which demonstrates excellent in vivo stability of the tracer.

TABLE 1
Metabolite analysis

| Time (p.i.) | Polar metabolites (%) | Apolar metabolites (%) | Parent (%) |
|---|---|---|---|
| 15 | 3.6 ± 0.3 | 8.9 ± 1.1 | 87.5 ± 0.8 |
| 45 | 5.6 ± 0.6 | 11.1 ± 1.9 | 83.3 ± 1.3 |

### Xenograft Selection and Sequencing

For assesment of tumor targetting potential three human NSCLC xenografts were selected. Each tumor type expresses EGFR with a different mutational status and has a different sensitivity to treatment with afatinib according to Li et al (oncogene 2008).

TABLE 2
Selected cell lines and associated mutational pattern.

| Cell Line | EGFR sequencing | IC₅₀¹ |
|---|---|---|
| A549 | WT | 1437 nM |
| H1975 | L858R/T790M | 327 nM |
| HCC827 | delE746-A750 (exon 19) | 0.2 nM |

¹Li et al.; Oncogene, 2008, 27, 4702.

### Immunohistochemical Staining

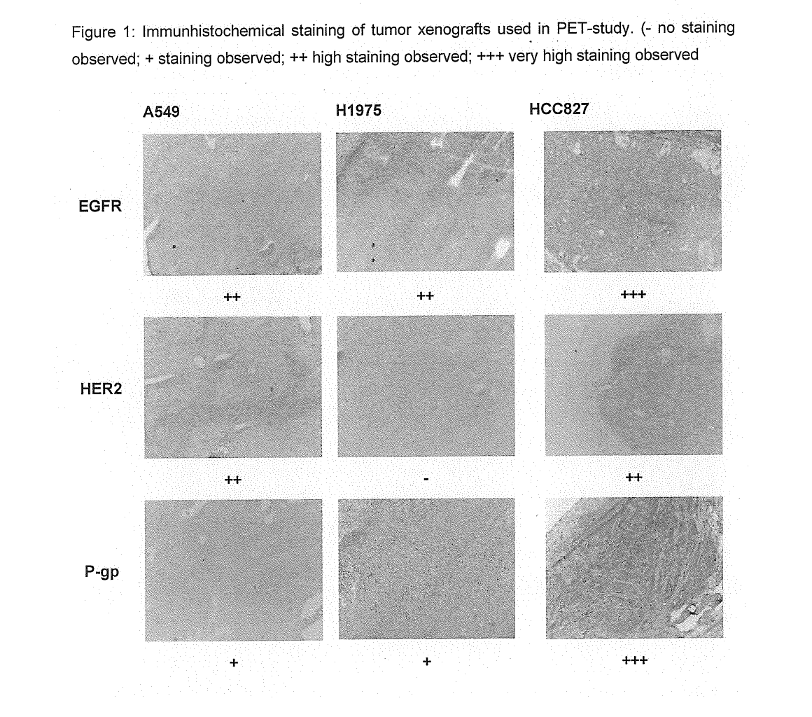

Sections of frozen xenografts (A549, HCC827) were immunostained for assessment of EGFR, HER-2 and P-gp expression. Antibodies were diluted in PBS (phosphate buffered saline) with 1% bovine serum albumin. EFGR was stained with cetuximab (Merck), HER2 with trastuzumab (Roche, Basel, Switzerland), and P-gp with rabbit polyclonal anti-P-gp (AB103477, ITK diagnostics BV, Uithoorn, the Netherlands). As secondary antibodies rabbit anti-human horseradish peroxidase (P0214, Dako, Glostrup, Denmark) or swine anti-rabbit horseradish peroxidase (P0217, Dako) were used. Cryosections (5 μm) of fresh frozen (tumor) tissue were air-dried and subsequently fixed with 2% paraformaldehyde in PBS for 10 minutes. Sections were blocked with normal rabbit serum (in case of trastuzumab or cetuximab) or with normal swine serum (in case of anti-P-gp) and subsequently stained with cetuximab 10 μg/ml (EGFR), trastuzumab 10 μg/ml (HER2) or anti-P-gp 5 μg/ml. Color development was performed with diaminobenzidine (DAB) and counterstaining was done with Hematoxiline (FIG. 1, reduced to monochrome).

Expression of the targets was confirmed via immunohistochemical staining and the mutations were confirmed by sequencing. Responsiveness to afatinib treatment is dictated by activating mutations commonly found in NSCLC patients with EGFR overexpression. Three clinically relevant cell lines were selected for evaluation of [¹⁸F]afatinib. For the non-responsive model the A549 cell line expressing WT EGFR was chosen, which has been reported to show no responsiveness to afatinib. As the responding cell line HCC827 was chosen expressing a mutated Variant of EGFR. This mutation concerns an exon 19 deletion variant conferring afatinib sensitivity in preclinical models. Third, the H1975 cell line was chosen harbouring a double mutation, first a sensitizing mutation of exon 20 (I858r) and secondly a mutation associated with acquired resistance to TKI treatment (T790M) All lines were further characterized using immunohistochemical staining for expression of the targets (EGFR and HER2). The results indicated that both cell lines express EGFR, however, the HCC827 does so to a higher extend (FIG. 1). HER2 is expressed by both cell lines to a similar extent. Overall, though immunohistochemical staining is a semi-quantitative method to determine target expression levels, the EGFR expression was most intense for HCC827, which is most sensitive to treatment with afatinib. Furthermore, the cells were stained for expression of P-gp, a well-known drug efflux transporter associated with drug resistance to tumors. All three lines express this efflux-pump, however, based on the obtained IHC stainings the HCC827 tumors showed the highest expression of P-gp.

### Biodistribution Studies

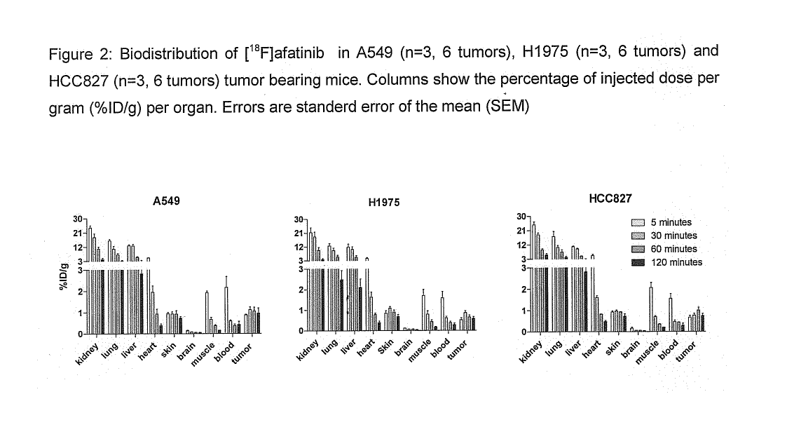

Nude mice (nu/nu) bearing two tumors (obtained by injection of A549, H1975 or HCC827 cells) of the same xenograft line on their left and right flank, received an injection of 15-MBq [¹⁸F]afatinib via the tail vein. The mice were sacrificed and dissected at 5, 30, 60 and 120 minutes post-injection. Blood, urine, skin, left tumor, right tumor, muscle, heart, lung, liver, kidney and brain were collected, weighed and counted for radioactivity in a Wallac Compugamma 1210 counter (n=3 for each time point). Biodistribution data are expressed as percentage of injected dose per gram (% ID/g) tissue for each organ (FIG. 2).

[¹⁸F]afatinib showed a rapid and high uptake in the metabolic organs (kidney and liver) as is more often observed for small molecule PET-tracers. Furthermore, high initial uptake was observed in well-perfused tissues like the heart and lungs. Due to the rapid excretion the blood level of the tracer was already quite low after 5 minutes p.i. (A549: 2.17% ID/g; H1975: 1.59% ID/g; HCC827: 1.56% ID/g). The investigated tumor types showed good initial uptake. Furthermore relevant background tissues such as blood and muscle were rapidly cleared of radioactivity, while the tumors showed good activity retention (around 1% ID/g remained in the tumor at 120 minutes p.i.). This led to moderate/high tumor-to-blood ratios (A549: 2.26 at 120 minutes p.i.; H1975: 2.11 at 120 minutes p.i.; HCC827: 2.59 at 120 minutes p.i.) and high tumor-to-muscle ratios (A549: 6.37 at 120 minutes p.i.; H1975: 3.48 at 120 minutes p.i.; HCC827: 3.83 at 120 minutes p.i.).

### PET-Imaging Studies

Dynamic PET imaging was performed on three cancer xenograft (A549, H1975 and HCC827) lines in nude mice. Each mouse (n=3) carried one tumor of the same cancer xenograft line, which were located on the left or right flank. Imaging was performed for a duration of 120 min using a double-LSO/LYSO layer high-resolution research tomograph (HRRT; CTI/Siemens, Knoxville, Tenn., USA). The mice were anesthetized with 4% and 2% isoflurane in 1 L·min⁻¹ oxygen for induction and maintenance, respectively. First, for attenuation and scatter correction, a transmission scan was acquired using a 740-MBq two-dimensional (2D) fan-collimated ¹³⁷Cs (662 keV) moving point source. Next, a dynamic emission scan was acquired immediately following administration (I.V. ocular plexus) of 4-6 MBq [¹⁸F]afatinib (SA 223±38 GBq/□mol) or 6-8 MBq of [¹¹C]erlotinib (SA: 184-587 GBq/□mol at end of synthesis) to each animal. Positron emission scans were acquired in list mode and rebinned into the following frame sequence: 10×60 s, 4×300 s, and 9×600 s. After 120 min, [¹⁸F]FDG was administered (I.V. ocular plexus) to the mice followed by scanning for another 60 min. Following corrections for decay, dead time, scatter and randoms, scans were reconstructed using an iterative 3D ordered-subsets weighted least-squares analysis (3D-OSWLS). Point source resolution varied across the field of view from approximately 2.3 to 3.2-mm full width at half maximum in the transaxial direction and from 2.5 to 3.4 mm in the axial direction. Post-filtering was not performed after reconstruction. The PET images were analyzed using the freely available AMIDE-software version 0.9.3 (A Medical Imaging Data Examiner). A box was drawn over the complete animal to obtain the image-derived injected dose (IDID). ROIs containing the tumor tissue as well as a reference area, which was drawn in the opposite flank of the animal containing the exact same tissue only devoid of tumor cells, were drawn using the [¹⁸F]FDG data. Subsequently the corresponding images obtained with [¹⁸F]afatinib or [¹¹C]erlotinib were overlayed. A time activity curve was plotted for both the tumor as well as the reference area. The images were smoothed using a gaussian (2 mm).

A thorough PET-imaging study was performed to evaluate the potential of [¹⁸F]afatinib as a TKI-PET-tracer. It was aimed to evaluate several factors that influence tracer uptake. To this end it was imaged with several different cold doses of the compound co-injected and also while blocking P-gp. P-gp is a drug-efflux transporter that actively removes xenobiotics from the cells. Immunohistochemical staining was used to determine the expression of this transporter and it was found that the HCC827 cells clearly expressed this pump to a high extend. Finally a comparative imaging study with [¹¹C]erlotinib was performed.

The selection of suitable background tissue is of vital importance. Initially the mice were xenografted with a tumor on each flank, however this left limited options to select suitable background tissue. Two important considerations for background tissue are: no vital organs should be present and it should contain well perfused normal tissue. The edge of the animal near the tail and the tail itself was selected for this purpose, however in both cases this led to extremely high tumor-to-background ratios that were not realistic. Therefore it was chosen to xenograft mice with only 1 tumor and use the same area in the other flank as background tissue (same slices/position in the PET scan). This solution led to good backgrounds and representative time-activity-curves (TAC's) which corresponded to the obtained PET-image.

PET-experiments were conducted on 3 mice of each cell line. The mice were anesthetized, cannulated and placed in the scanner. In the case of a blocking experiment, tariquidar was administered 20 minutes prior to the start of the scan. First the mice were administered with circa 4-6 MBq of [¹⁸F]afatinib or 6-8 MBq of [¹¹C]erlotinib, followed by a dynamic scan of 120 minutes or 90 minutes respectively. Next the mice were checked and administered 5 MBq of [¹⁸F]FDG followed by dynamic scanning for 60 min. After scanning the mice were allowed to recover.

PET-images were processed using AMIDE (Version 0.9.2). The FDG scan was used to determine the Region Of Interest (ROI) for the tumor and the background tissue. The [¹⁸F]afatinib scan was overlaid and a total dose box was drawn over the entire animal. The injected dose per gram was derived from these ROIs (counts in ROI) providing an image derived injected dose for the tumor and the background for each of the animals.

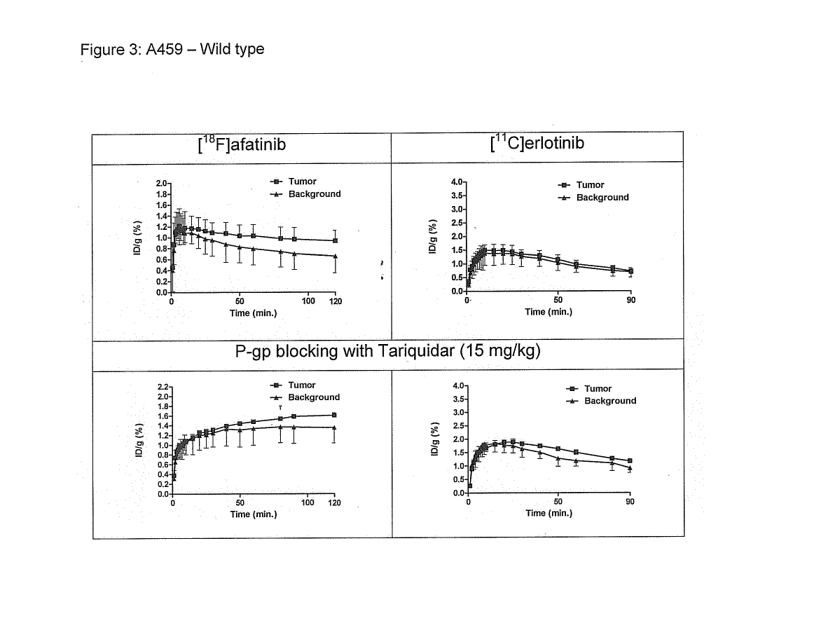

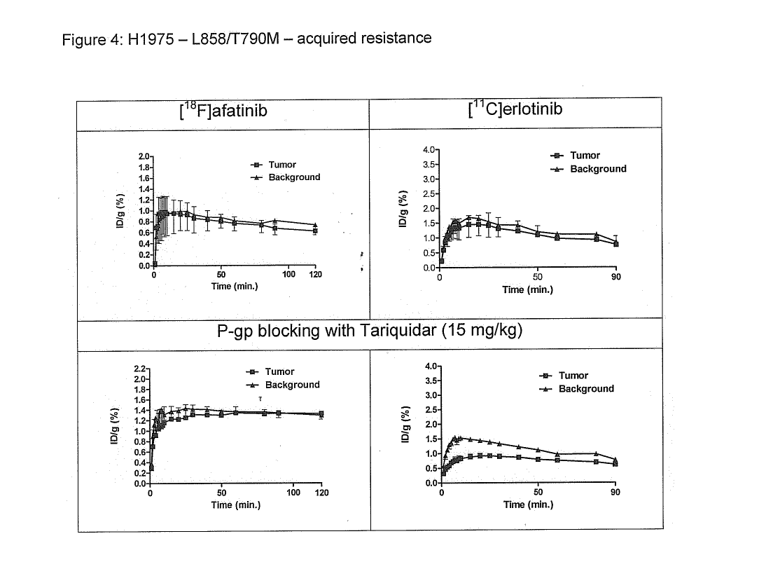

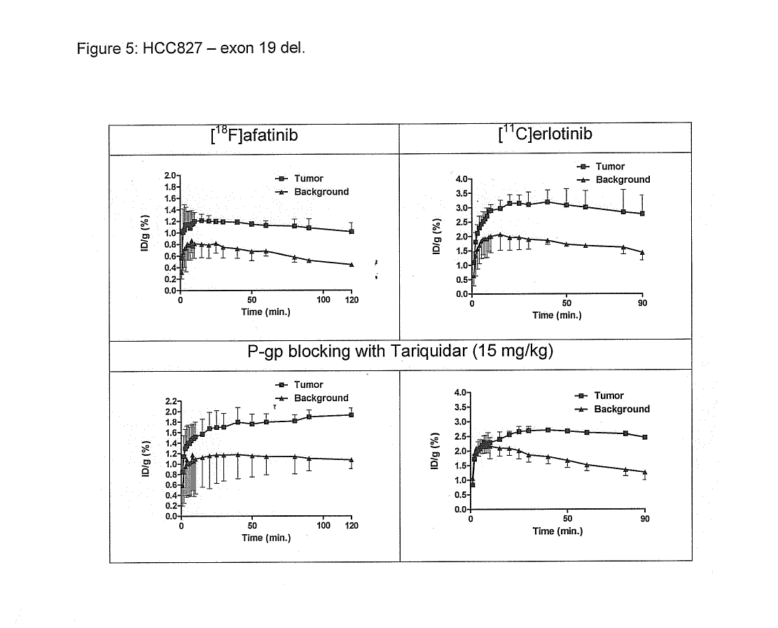

The results of the experiments are grouped per cell line (FIGS. 3 to 5). The left 2 panels are the [¹⁸F]afatinib time activity curves (TAC) (top: no block, bottom: blocked with tariquidar) and the right are the [¹¹C]erlotinib scans (top: no block, bottom: blocked with tariquidar).

[¹⁸F]afatinib shows similar imaging propertips, when compared to [¹¹C]-erlotinib when no blocking is performed. Uptake is found in the sensitive cell line (HCC827, FIG. 5) and while the absolute amount of activity (% ID/g) is lower than [¹¹C]-erlotinib, the tumor-to-background ratio is higher (2.3 at 120 min p.i. vs. 1.9 at 90 min p.i.). The difference in absolute uptake is most likely related to the difference in kinetics between the two tracers. [¹⁸F]afatinib accumulation is quite rapid, reaching the maximum after circa 3-4 minutes, while [¹¹C]erlotinib keep accumulating up to about 15-20 minutes. The insensitive cell-lines (A549 and H1975) show a similar trend for both tracers as well, with hardly an increase of uptake in the tumors compared to the background. This result shows that [¹⁸F]afatinib can differentiate between treatment responsive tumors in the same manner as [¹¹C]erlotinib.

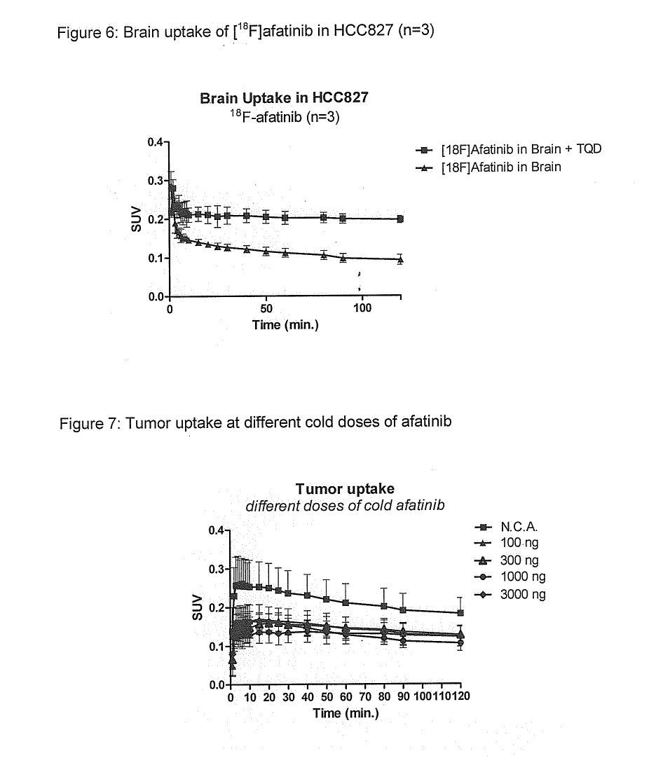

An important difference is observed when P-gp is blocked during scanning. To confirm the actual blocking of P-gp a region of interest was drawn on the brain and the uptake was determined for [¹⁸F]afatinib. This showed that when P-gp was active [¹⁸F]afatinib is washed out from the brain and under blocking conditions remains in the brain (FIG. 6).

Having confirmed the blocking of P-gp we performed the same scanning experiment while blocking. The most important effect was that absolute tumor uptake increased for both tracers in almost all cell lines (with the exception of H1975 for [¹¹C]erlotinib) confirming that both tracers are substrates and thus pumped out during regular PET-experiments. The difference in the sensitive HCC827 cells between the tumor and background increases even more for both tracers, which is accordance with the expression of PgP on this cell line. An important difference can be observed between [¹⁸F]afatinib and [¹¹C]erlotinib as after P-gp blocking [¹⁸F]afatinib hardly shows any washout, most likely due to the irreversible binding to the ATP binding site. This result might suggest that P-gp efflux could be quicker than the irreversible binding to EGFR, as without P-gp blocking this trend is not observed.

Lastly we also imaged in the presence of varying amounts of cold afatinib added to the [¹⁸F]afatinib injection in an attempt to mimic a therapeutic dose (FIG. 7). This showed that already when adding a cold dose of 100 ng tumor uptake was reduced to background levels. Showing that imaging of the tumor should be done at high specific activity.

### PREPARATION OF INTERMEDIATES

### List of Abbreviations

bm—broad multiplet
BOP—benzotriazole-1-yl-oxy-tris-(dimethylamino)-phosphonium hexafluorophosphate
bs—broad singlet
d—doublet
DBU—2,3,4,6,7,8,9,10-octahydropyrimido[1,2-a]azepine
DIPA—Diisopropylethylamine
DMF—dimethylformamide
ESI—electron spray ionization
EtOAc—ethylacetate
g—gram
h—hour(s)
HPLC—high performance liquid chromatography
HR-MS—high resolution mass spectrometry
Hz—Hertz
M—molar
m—multiplet
m/z—mass to charge ratio
MeCN—acetonitrile
MeOH—methanol
mg—miligram
ml—mililiter
mm—milimeter
mmol—milimole
NMP—N-methylpyrrolidine
NMR—Nuclear Magnetic Resonance
q—quartet
s—singlet
semi-prep—semi-preparative
SA—specific activity
t—triplet
TLC—thin layer chromatography
v/v—volume ratio
ml—microliter

The preparation of precursor compound (4) is described in scheme 1:

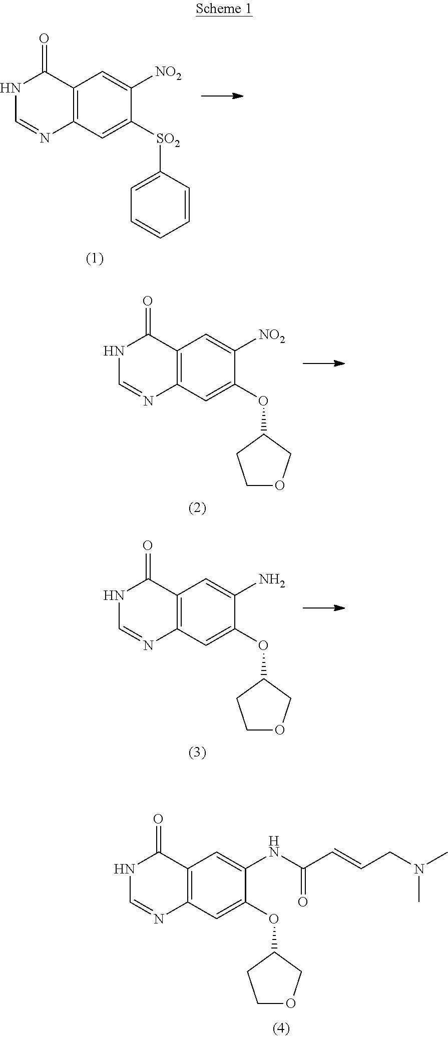

### Example 1

### 6-Nitro-7-(phenylsulfonyl)quinazolin-4(3H)-one (1)

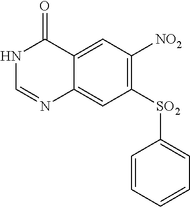 [SMILES: O=c1[nH]cnc2cc(S(=O)(=O)c3ccccc3)c([N+](=O)[O-])cc12]

7-chloro-6-nitroquinazolin-4(3H)-one (2 g, 8.87 mmol) and benzenesulfinic acid sodium salt (1.455 g, 8.87 mmol) were suspended in DMF (30 mL) and heated to 90° C. for 6 h. The reaction mixture was diluted with H₂O (30 mL) and the precipitate was collected by vacuum filtration. The resulting solid was dried in vacuo to obtain 6-nitro-7-(phenylsulfonyl)quinazolin-4(3H)-one. ¹H-NMR (500.23 Mhz, [D₆]DMSO) □: 12.97 (bs, 1H), 8.61 (s, 1H) 8.52 (s, 1H), 8.42 (s, 1H), 8.05 (d, J=7.66 Hz, 2H), 7.78 (t, J=7.41 Hz, 1H), 7.70 (t, J=7.81 Hz, 2H); ¹³C-NMR (125.78 Mhz, [D₆]DMSO) □: 159.7, 151.7, 150.2, 144.7, 140.0, 138.3, 135.15, 132.1, 130.2, 128.6, 127.2, 124.8; HR-MS (ESI, 4500V): m/z calculated for C₁₄H9N₃NaO₅S⁺ (M+Na⁺): 354.0155. found: 354.0146.

### Example (2)

### (S)-6-nitro-7-((tetrahydrofuran-3-yl)oxy)quinazolin-4(3H)-one (2)

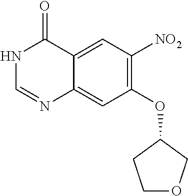 [SMILES: O=c1[nH]cnc2cc(O[C@H]3CCOC3)c([N+](=O)[O-])cc12]

To a solution of 6-nitro-7-(phenylsulfonyl)quinazolin-4(3H)-one (2.0 g, 6.04 mmol) and (S)-tetrahydrofuran-3-ol (0.627 mL, 7.85 mmol), in tert-butanol/DMF (25 mL/5 mL) stirred under argon, was added dropwise potassium tert-butoxide (1M in THF, 21.73 mL, 21.73 mmol) at 20° C. The mixture was stirred for 16 h at 200° C. and then at 45° C. until TLC indicated full consumption of 6-nitro-7-(phenylsulfonyl)quinazolin-4(3H)-one. All volatiles were removed in vacuo to obtain the crude product which was purified by flash column chromatography (MeOH/EtOAc, 5:95 v/v) to afford (S)-6-nitro-7-((tetrahydrofuran-3-yl)oxy)quinazolin-4(3H)-one. ¹H-NMR (500.23 Mhz, [D₆]DMSO) □: 12.55 (bs, 1H), 8.50 (s, 1H), 8.22 (s, 1H), 7.40 (s, 1H), 5.41 (t, J=4.64 Hz, 1H), 3.95 (bm, 4H), 2.31 (sextet, J=7.86, 13.90, 22.02 Hz, 1H), 2.03 (q, J=6.90, 12.45 Hz, 1H); ¹³C-NMR (125.78 Mhz, [D₆]DMSO) □: 160.2, 154.4, 153.4, 149.4, 139.5, 124.5, 115.9, 112.3, 80.5, 72.5, 67.0, 32.9; HR-MS (ESI, 4500V): m/z calculated for C₁₂H₁₁N₃NaO₅⁺ (M+Na⁺): 300.0591. found: 300.0573.

### Example (3)

### (S)-6-amino-7-((tetrahydrofuran-3-yl)oxy)quinazolin-4(3H)-one (3)

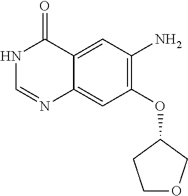 [SMILES: Nc1cc2c(=O)[nH]cnc2cc1O[C@H]1CCOC1]

To a refluxing solution (110° C.) of (S)-6-nitro-7-((tetrahydrofuran-3-yl)oxy)quinazolin-4(3H)-one (1.2 g, 4.33 mmol) and acetic acid (1.98 mL, 34.6 mmol) in ethanol/water (27.5 mL, 10:1, v/v) was added iron powder (967 mg, 17.31 mmol), the mixture was allowed to reflux (110° C.) for 20 minutes. Then the mixture was allowed to cool to 20° C. and applied to a celite filter and eluted with ethanol, the product containing fractions were concentrated to obtain the crude product and purified by flash column chromatography (MeOH/EtOAc, 5:95, v/v) to afford (S)-6-amino-7-((tetrahydrofuran-3-yl)oxy)quinazolin-4(3H)-one. ¹H-NMR (500.23 Mhz, [D₆]DMSO) □: 12.78 (bs, 1H), 7.79 (s, 1H), 7.23 (s, 1H), 6.93 (s, 1H), 5.31 (s, 2H), 5.17 (t, J=4.6 Hz, 1H), 3.96 (m, 1H), 3.88 (m, 2H), 3.77 (m, 1H), 2.26 (sextet, J=7.44, 13.70, 21.6 Hz, 1H), 2.07 (q, J=6.8, 12.2 Hz, 1H); ¹³C-NMR (125.78 Mhz, [D₆]DMSO) □: 160.7, 150.4, 141.9, 141.8, 139.1, 117.3, 108.7, 106.6, 78.5, 72.8, 67.1, 33.1; HR-MS (ESI, 4500V): m/z calculated for C₁₂H₁₃N₃NaO₃+(M+Na⁺): 270.0849. found: 270.0832.

### Example (4)

### (S,E)-4-(dimethylamino)-N-(4-oxo-7-((tetrahydrofuran-3-yl)oxy)-3,4-dihydroquinazolin-6-yl)but-2-enamide (4)

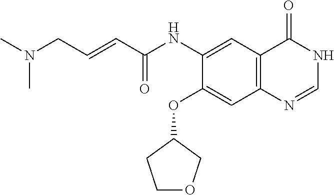

To a suspension of commercially available (2E)-4-(dimethylamino)but-2-enoic acid hydrochloride (50 mg, 0.4 mmol) in THF (3 mL) containing a catalytic amount of DMF (0.05 mL) under an inert atmosphere was added oxalylchloride (31.9 DL, 0.36 mmol) at 0° C. When foaming ceased the mixture was heated to 25° C. and kept at this temperature for 90 minutes. The mixture was then cooled to 0° C. and a solution of (S)-6-amino-7-((tetrahydrofuran-3-yl)oxy)quinazolin-4(3H)-one (50 mg, 0.2 mmol) in N-methylpyrrolidine (1 mL) was added in a stream. The mixture was allowed to come to room temperature slowly, then anhydrous diisopropylethylamine (106 □I, 1.2 mmol) was added. When consumption of the starting amine was observed on TLC the reaction was quenched by the addition of aqueous NaHCO₃ (1 mL). The volatiles were removed by rotary evaporation and the remainder was purified by flash column chromatography (Gradient: MeOH:EtOAc=5:95, v/v to MeOH:EtOAc=20:80, v/v) to afford (S,E)-4-(dimethylamino)-N-(4-oxo-7-((tetrahydrofuran-3-yl)oxy)-3,4-dihydroquinazolin-6-yl)but-2-enamide. ¹H-NMR (500.23 Mhz, [D₆]DMSO) □: 12.22 (s, 1H), 9.32 (s, 1H), 8.85 (s, 1H), 8.00 (s, 1H), 7.13 (s, 1H), 6.76 (m, 1H), 6.64 (d, 1H), 5.76 (t, J=5.25 Hz, 1H), 3.95 (bm, 3H), 3.76 (sextet, J=4.9, 8.0, 13.0 Hz, 1H), 3.11 (d, J=5.0 Hz, 2H), 2.30 (sextet, J=7.2, 13.7, 21.2 Hz, 1H), 2.20 (s, 6H), 2.16 (m, 1H); ¹³C-NMR (125.78 Mhz, [D₆]DMSO) □: 164.1, 160.6, 153.7, 147.2, 145.7, 128.0, 126.9, 117.9, 116.2, 109.2, 79.5, 72.5, 67.2, 60.11, 45.5, 32.9. HR-MS (ESI, 4500V): m/z calculated for C₁₈H₂₃N₄O₄⁺ (M+H⁺): 359.1714. found: 359.1770.

### Preparation of Final Compound

The preparation of compound (5) is described in scheme 2:

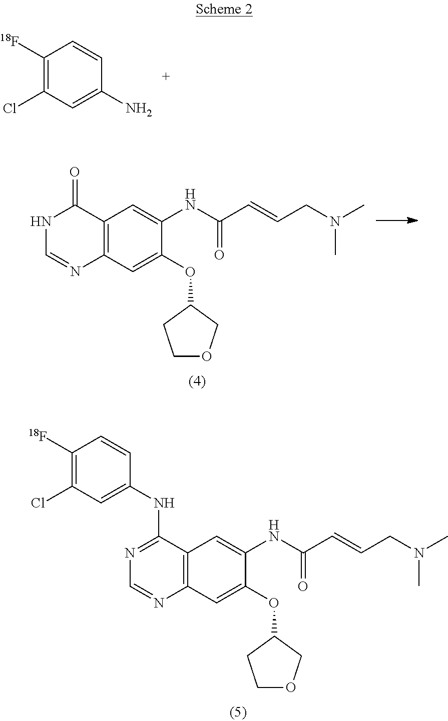

### Example 5

### [¹⁸F](S,E)-N-(4-((3-chloro-4-fluorophenyl)amino)-7-((tetrahydrofuran-3-yl)oxy)quinazolin-6-yl)-4-(dimethylamino)but-2-enamide (5)

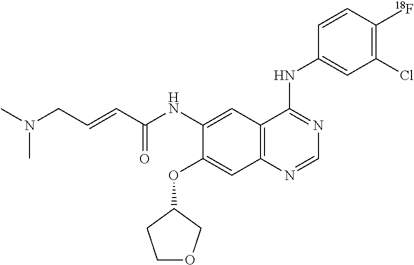

1-50 Gbq of 3-Chloro-4-[¹⁸F]fluoroaniline (J Label Compd Radiopharm 2005, 48, 829-843) is added to a solution of (S,E)-4-(dimethylamino)-N-(4-oxo-7-((tetrahydrofuran-3-yl)oxy)-3,4-dihydroquinazolin-6-yl)but-2-enamide (2 mg), benzotriazole-1-yl-oxy-tris-(dimethylamino)-phosphonium hexafluorophosphate (BOP, 5 mg), 2,3,4,6,7,8,9,10-octahydropyrimido[1,2-a]azepine (DBU, 2.5 □I) in anhydrous N-methylpyrrolidine (NMP, 1 mL). The thus obtained mixture is heated to 120° C. for 30 minutes after which it is cooled to 20° C. and quenched by the addition of water (1 mL) and purified by preparative HPLC chromatography (column: Altima-C18, 5 uM, 10*250 mm semi-prep, eluent: MeCN/H₂O/DIPA, 45/

55/0.1, v/v/v, flow: 4 ml/min), retention time of the product is 23-26 minutes.

### Formulation:

The collected fraction (23-26 minutes) of the preparative HPLC containing the product was diluted with 50 mL of water and the total mixture was passed over a tC18 waters seppak cartridge. The cartridge was then washed with 20 mL of sterile water after which the product was eluted from the cartridge with 1.5 mL of sterile 96% ethanol. The ethanol was diluted to 10 volume percent with sterile saline and the complete solution was filtered over a MILLEX GV 0.22 μm filter into a sterile 20 mL capped vial.

Analysis of the product was performed by analytical HPLC (Column: Platinum-C18, 5 uM, 250×4.6 mm analytical column, eluent: MeCN/H₂O/DIPA, 60/40/0.1, v/v/v, flow: 1 ml/min), retention time of the product is 9-11 minutes.

1. A fluorine-18 labeled compound of formula (I) 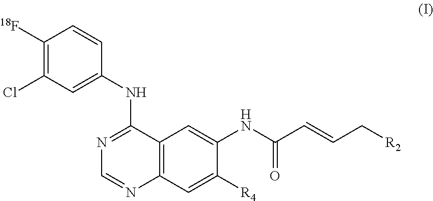
wherein
R² represents dimethylamino-, diethylamino-, morpholino-, [1,4]oxazepan-4-yl-;
R⁴ represents tetrahydrofuran-3-yl-oxy-, tetrahydrofuran-2-yl-methoxy-, tetrahydrofuran-3-yl-methoxy-, tetrahydropyran-4-yl-oxy-, or tetrahydropyran-4-yl-methoxy-.

2. A radiolabeled compound according to claim 1, wherein
R² represents dimethylamino-.

3. A radiolabeled compound according to claim 1, wherein
R⁴ represents

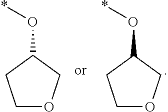

4. The radiolabeled compound according to claim 1 selected from the group consisting of

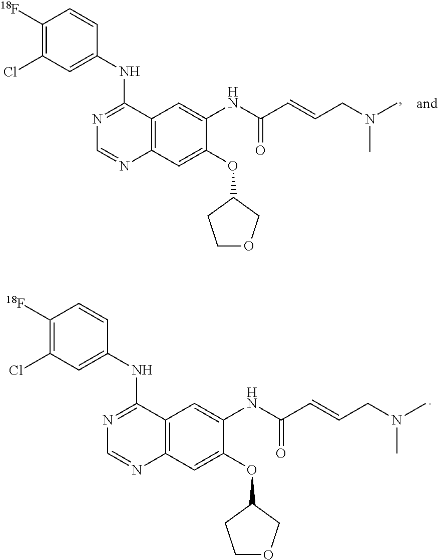

5. A Intermediate compound of formula (II) 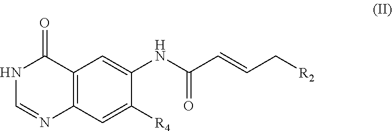
wherein
R² and R⁴ are defined as in claim 1.

6. A method for preparing a radiolabeled compound according to claim 1, said method comprising:
reacting radiolabeled 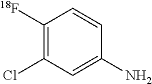
with a compound of formula (II) 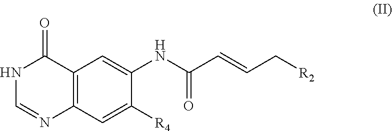
wherein
R² and R⁴ are defined as in claim 1,
and isolating the resulting compound of formula (I).

7. A method for preparing the compound of formula (II) according to claim 5, said method comprising:
reacting a compound of formula (III) 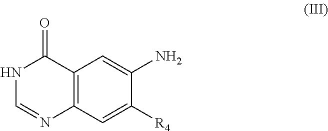
wherein
R⁴ is defined as in claim 5,
with a compound of formula (IV) 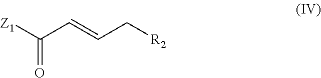
wherein
R² is defined as in claim 5, and Z₁ is a leaving group or a hydroxy group.

8. A method of using a radiolabeled compound according to claim 1, or a pharmaceutically acceptable salt thereof, for in in vivo diagnosis, tumor imaging or patient stratification on the basis of mutational status of EGFR (erbB1).

9. A radiopharmaceutical composition comprising a radiolabeled compound of formula (I) according to claim 1, or a pharmaceutically acceptable salt thereof, optionally together with one or more inert carriers and/or diluents.

10. A method for in vivo diagnosis, tumor imaging or patient stratification on the basis of mutational status of EGFR (erbB1), the method comprising administering a radiolabeled compound according to claim 1, or a pharmaceutically acceptable salt thereof, to a patient prior to undergoing positron emission tomography (PET) or single photon emission computed tomography (SPECT) to assess the mutational status of the tyrosine kinase (TK) present in the tumors of the patient.
